(19) United States
(12) Patent Application Publication
(10) Pub. No.: US 2015/0000371 A1
(43) Pub. Date: Jan. 1, 2015

(54) BEVERAGE DIAGNOSTIC AND PRESERVATION DEVICES AND METHODS

(71) Applicants: Russell W. Greene, Atlanta, GA (US); Daniel B. Lipton, Atlanta, GA (US); David J. Trettin, Atlanta, GA (US); Trent J. Kahute, Atlanta, GA (US); James H. Wood, San Antonio, TX (US); Jerome A. Helffrich, San Antonio, TX (US); David J. Postell, San Antonio, TX (US); Bob Vanecek, San Antonio, TX (US)

(72) Inventors: Russell W. Greene, Atlanta, GA (US); Daniel B. Lipton, Atlanta, GA (US); David J. Trettin, Atlanta, GA (US); Trent J. Kahute, Atlanta, GA (US); James H. Wood, San Antonio, TX (US); Jerome A. Helffrich, San Antonio, TX (US); David J. Postell, San Antonio, TX (US); Bob Vanecek, San Antonio, TX (US)

(21) Appl. No.: 14/272,495

(22) Filed: May 7, 2014

Related U.S. Application Data
(60) Provisional application No. 61/820,429, filed on May 7, 2013.
(60) Provisional application No. 61/867,236, filed on Aug. 19, 2013.
(60) Provisional application No. 61/902,561, filed on Nov. 11, 2013.

(51) Int. Cl. G01N 33/14; G01N 21/27; G01N 33/00

(52) U.S. Cl. CPC G01N 33/146; G01N 33/0042; G01N 33/0065; G01N 21/27
USPC 73/19.1

(57) ABSTRACT

Beverage diagnostic and preservation devices and methods are described. In several exemplary embodiments, one or more of the devices are used to detect the freshness of, and/or preserve, wine in a container. The devices can receive inputs from one or more sensors when determining the freshness of wine in a container.

### CROSS-REFERENCE TO RELATED APPLICATIONS

This application claims the benefit of U.S. Provisional Patent Application No. 61/820,429, filed May 7, 2013; U.S. Provisional Patent Application No. 61/867,236, filed Aug. 19, 2013; and U.S. Provisional Patent Application No. 61/902,561, filed Nov. 11, 2013; each of which is hereby incorporated by reference in its entirety.

### BACKGROUND

This disclosure relates in general to beverage diagnostic and preservation devices, and, in particular, to beverage diagnostic and preservation devices configured to couple to a beverage container, such as a wine bottle.

### BRIEF DESCRIPTION OF THE DRAWINGS

FIG. 1 is a side view of a beverage diagnostic device, according to an exemplary embodiment.

FIGS. 2-4 are side sectional views of the beverage diagnostic device of FIG. 1.

FIG. 5 is a side sectional view of a beverage diagnostic device in a closed position, according to another exemplary embodiment.

FIG. 6 is a side sectional view of the beverage diagnostic device of FIG. 5 in an open position.

FIG. 7 is a top view of the beverage diagnostic device of FIG. 5.

FIG. 8 is a top sectional view of the beverage diagnostic device of FIG. 5.

FIG. 9 is a side view of the beverage diagnostic device of FIG. 5 in a closed position.

FIG. 10 is another side view of the beverage diagnostic device of FIG. 5 in an open position.

FIGS. 11 and 12 are additional side sectional views of the beverage diagnostic device of FIG. 5

FIG. 13 is a side view of a beverage diagnostic device, according to another exemplary embodiment.

FIG. 14 is a side sectional view of the beverage diagnostic device of FIG. 13.

FIG. 15 is a side sectional view of the beverage diagnostic device of FIG. 13 in an open position.

FIG. 16 is a side sectional view of the beverage diagnostic device of FIG. 13 in a closed position.

FIG. 17 is a top view of the beverage diagnostic device of FIG. 13.

FIG. 18 is a top sectional view of the beverage diagnostic device of FIG. 13.

FIG. 19 is a diagrammatic illustration of a wine spout according to an exemplary embodiment.

FIG. 20 is a perspective view of a wine spout, according to an exemplary embodiment.

FIG. 21 includes perspective views of a wine spout, according to another exemplary embodiment.

FIG. 22 is an elevational view of a wine bottle stopper, according to an exemplary embodiment.

FIG. 23 is an exploded view of the wine bottle stopper of FIG. 22, according to an exemplary embodiment.

FIG. 24 is a perspective view of a portion of the wine bottle stopper of FIGS. 22 and 23, according to an exemplary embodiment.

FIG. 25 is a perspective view of the wine bottle stopper of FIGS. 22-24, according to an exemplary embodiment.

FIG. 26 includes elevational views of wine bottle stoppers according to respective exemplary embodiments.

FIG. 27 includes views of wine bottle stoppers according to respective exemplary embodiments.

FIG. 28 is an elevational view of a wine bottle stopper according to another exemplary embodiment, the wine bottle stopper including one or more wine spout features, aspects, or elements.

FIG. 29 is an exploded view of the wine bottle stopper of FIG. 28, according to an exemplary embodiment.

FIG. 30 includes views of the wine bottle stopper of FIGS. 28 and 29, according to respective exemplary embodiments.

FIG. 31 includes elevational and partially exploded views of a wine bottle stopper according to yet another exemplary embodiment, the wine bottle stopper including one or more wine preservation features, aspects, or elements.

FIG. 32 includes a perspective view of the wine bottle stopper of FIG. 31.

FIG. 33 includes views of wine bottle stoppers according to still yet other exemplary embodiments, each of the wine bottle stoppers including one or more wine preservation features, aspects, or elements.

FIG. 34 includes views of a system according to an exemplary embodiment, the system including a wine bottle stopper and a preservation device.

FIG. 35 is a perspective view of the system of FIG. 34, according to an exemplary embodiment.

FIG. 36 is a perspective view of a system according to an exemplary embodiment, the system including the system of FIGS. 34 and 35, as well as additional wine bottle stoppers.

FIG. 37 shows test results for an experiment in which sulfur dioxide (SO₂) and hydrogen sulfide (H₂S) was measured in a bottle of red wine and a bottle of white wine using a gas chromatograph detector with an electron capture detector (ECD).

FIG. 38 shows spectral data for the experiment introduced in FIG. 37. The x-axis shows wavelength in nanometers, and the y-axis shows absorbance.

### DETAILED DESCRIPTION

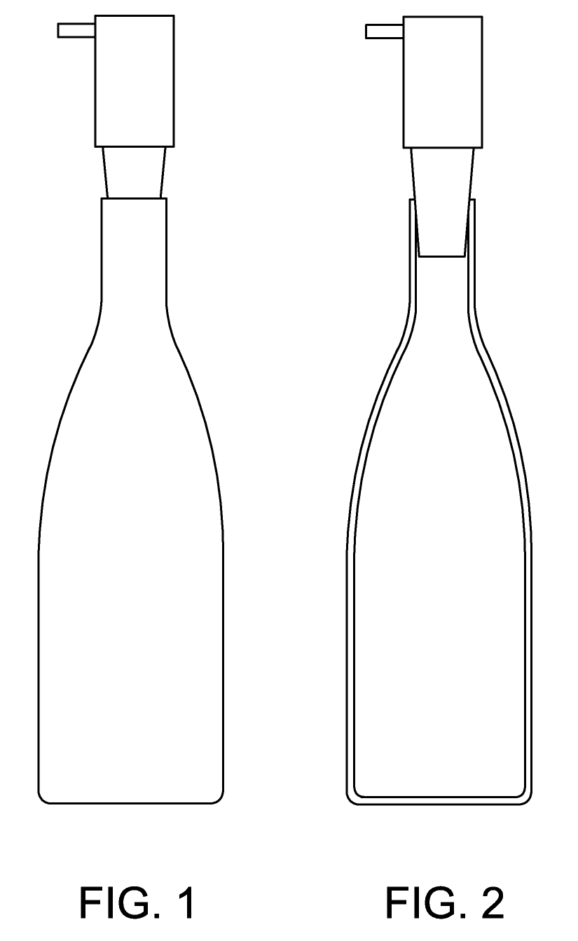

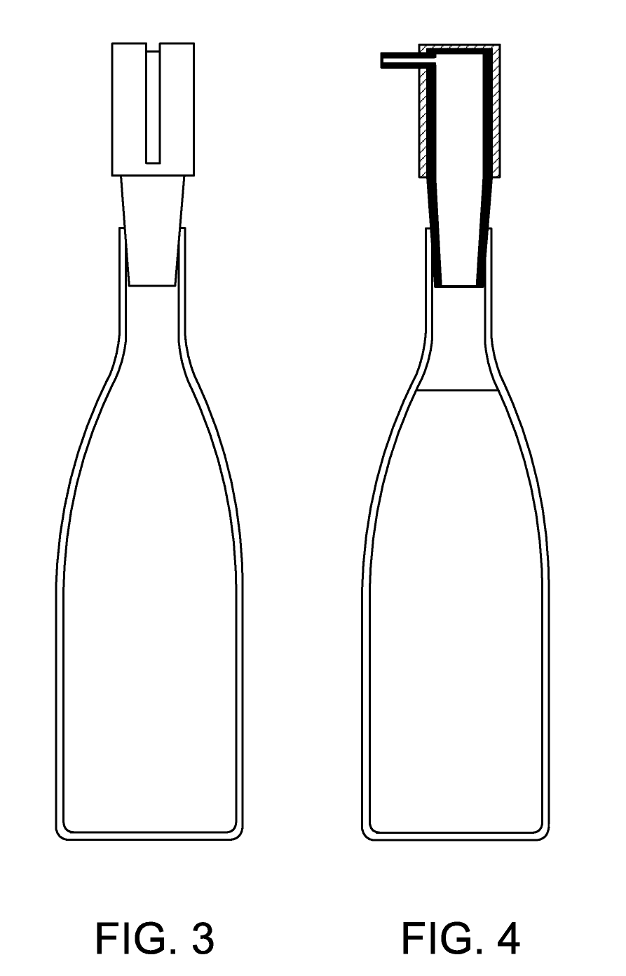

In an exemplary embodiment, as illustrated in FIGS. 1-4, a beverage diagnostic device is coupled to a beverage container, such as a wine bottle, in which wine or any fluid may be stored. In one embodiment, the device includes a body, the body having a lower portion, an upper portion, and a fluid chamber formed therein. In one embodiment, the lower portion of the device is coupled to an upper portion of a wine bottle using a press fit. In one embodiment, an outside diameter of the lower portion of the device is smaller than an inside diameter of the upper portion of the wine bottle. In one embodiment, the lower portion of the device is formed from a rubber material, plastic material, or any other type of material. In one embodiment, the fluid chamber includes a fluid passage extending from the lower portion of the device to a spout located on the upper portion of the device. In one embodiment, the fluid passage allows fluid to flow from the container, through the lower portion of the device and out of the spout. In one embodiment, the device includes a freshness sensor. In one embodiment, the freshness sensor is a timer. In one embodiment, the timer is a digital timer, a mechanical timer, an electrical timer or any combination thereof. In one embodiment, coupling the device to the container activates the timer so that the timer begins recording a first time, where the first time is a duration. In one embodiment, the device includes a timer activator. In one embodiment, the timer activator is in contact with the inner diameter of the container and detects the coupling of the device to the container. In one embodiment, the timer activator activates the timer. In one embodiment, the timer activator is the timer. In one embodiment, the first time is a period of time beginning when the device is coupled to the container. In one embodiment, the device includes a timer display so that the first time is displayed to a user. In one embodiment, the timer display is located on the upper portion of the device. In one embodiment, the timer display has a digital screen displaying a digital counter. In one embodiment, the first time extends from installation of the device into until the device is removed or detached from the container. In one embodiment, the timer activator detects the removal or decoupling of the device from the container. In one embodiment, upon the timer activator's detection of the removal or decoupling of the device from the container, the timer's first time is set to zero. In one embodiment, coupling the device to a second container begins the recording of a second time, similarly to the first time. In one embodiment, the device may be coupled to the container to record the freshness of any fluid within the container. For example, after opening a bottle of wine, the device may be coupled to the bottle of wine, with the timer displaying how long the timer has been coupled to the bottle of wine. That is, the freshness of any wine contained in the container correlates to the period of time that has passed since the bottle of wine has been opened. When the device is coupled to the wine bottle directly after it is opened, the freshness of the wine is measured by the timer.

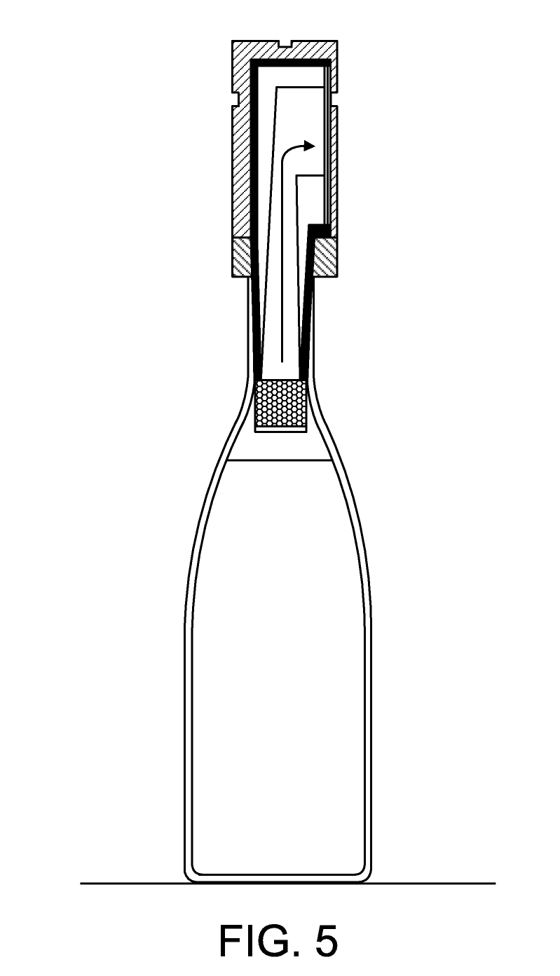

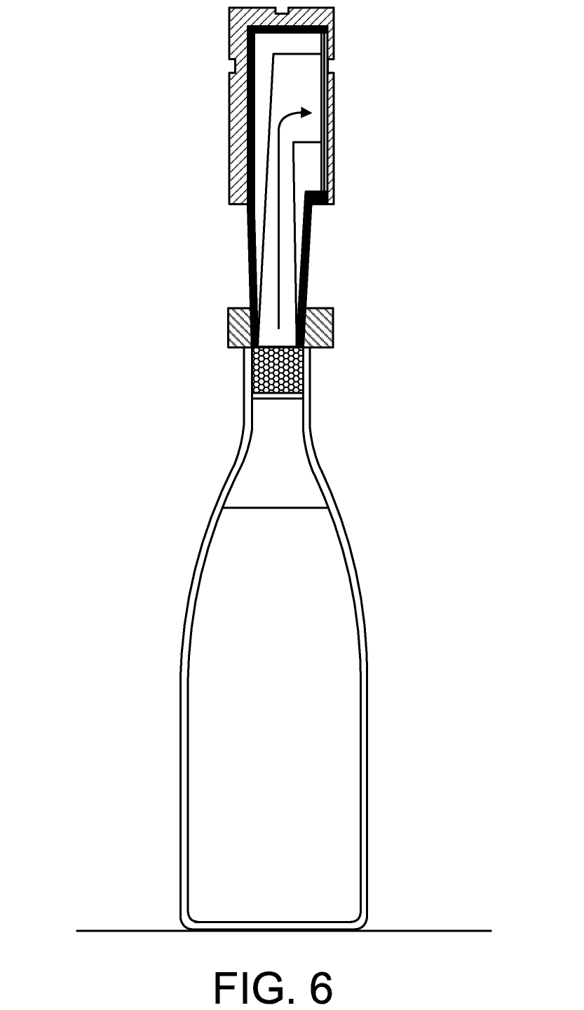

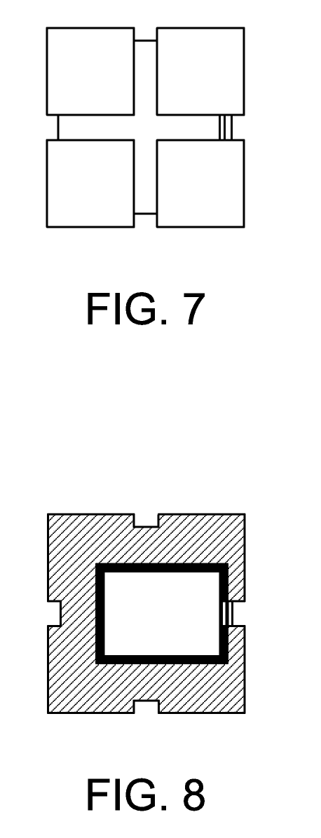

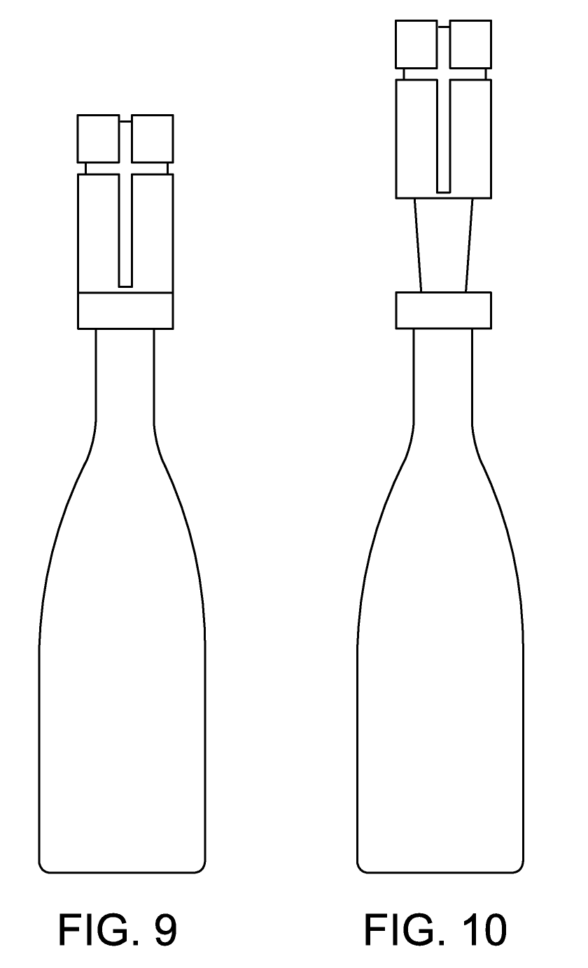

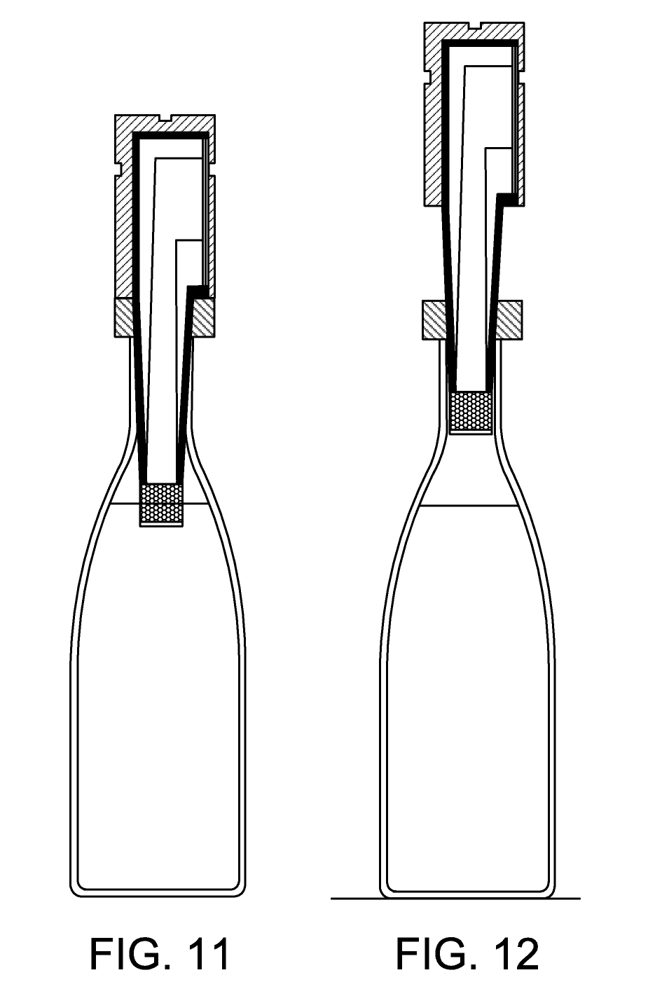

In another exemplary embodiment, as illustrated in FIGS. 5-12, a beverage diagnostic device has a freshness sensor that measures the activity of the (solvated) hydrogen ions and/or the hydrogen ion concentration (pH). In one embodiment, the freshness sensor includes an element that measures the pH of the fluid stored in the container. In one embodiment, the freshness sensor is a pH strip. In one embodiment, the element can measure the pH of the fluid stored in the container by contacting vapors, fumes, or chemicals emanating from the fluid or by contacting the fluid itself. In one embodiment, and as shown in FIG. 5, the device includes a band located above the lower portion of the device. In one embodiment, the band is configured to couple to an outer diameter of the upper portion of the wine bottle. In one embodiment, the lower portion of the device moves relative to the band to move the device between an open position and a closed position. In an exemplary embodiment, the element is a filter that is coupled to the lower portion of the device. In one embodiment, and as shown in FIG. 11, the filter contacts the fluid when the device is in the closed position. In one embodiment, vapor or fumes are forced through the filter and into the fluid chamber when the device moves between an open position and a closed position. In one embodiment, fluid is drawn into the filter when the device is in the closed position. For example, the fluid could be drawn into the filter due to a wicking force or capillary action associated with the filter. In one embodiment, the fluid chamber forms a contact chamber in the upper portion of the device. In one embodiment, a freshness indicator is coupled to an inner surface of the contact chamber. In one embodiment, a pocket or opening is formed in the contact chamber of the device so that a freshness indicator that is placed in the opening is visible by a user. In one embodiment, the freshness indicator is coupled to the filter so that the pH measurement measured by the filter is displayed on the freshness indicator. In another embodiment, the element is located in the contact chamber. In one embodiment, the element is a pH test strip that is configured to be placed in the opening or pocket. In one embodiment, the element measures the pH of the fluid when the vapor or fumes from the fluid are forced through the filter and into the contact chamber.

In one embodiment, the pH measurement displayed on the freshness indicator may be indicated through the use of text, color, etc. For example, the freshness indicator might display a green color if the pH of the fluid is within a first range, a purple color if the pH of the fluid is within a second range, and a red color if the pH of the fluid is within a third range. In one embodiment, the color green is displayed when the wine is most fresh, the color purple is displayed when there is roughly 3-4 days of shelf life left, and the color red is displayed when the wine is perceived to be spoiled.

In one embodiment, changing the position of the device, from the open position to the closed position creates a vacuum within the container, and/or seals the container, so that the freshness of the fluid within the container is preserved or the rate of spoliation of the fluid is reduced.

In one embodiment, the device preserves the freshness of the fluid. In one embodiment, the device preserves the freshness of the fluid by preventing oxygen from entering the container.

In one embodiment, the device detects, provides an indication of a beverage's freshness, and/or provides an indication of the remaining period of time that the beverage will be fresh. In another embodiment, freshness of a beverage is determined through the monitoring and/or detection of freshness factors including oxidation levels; sulfur containing compounds; sulfur dioxide; and/or the beverage's overall “volatile acidity” by the freshness indicator. In one embodiment, the beverage is a fluid.

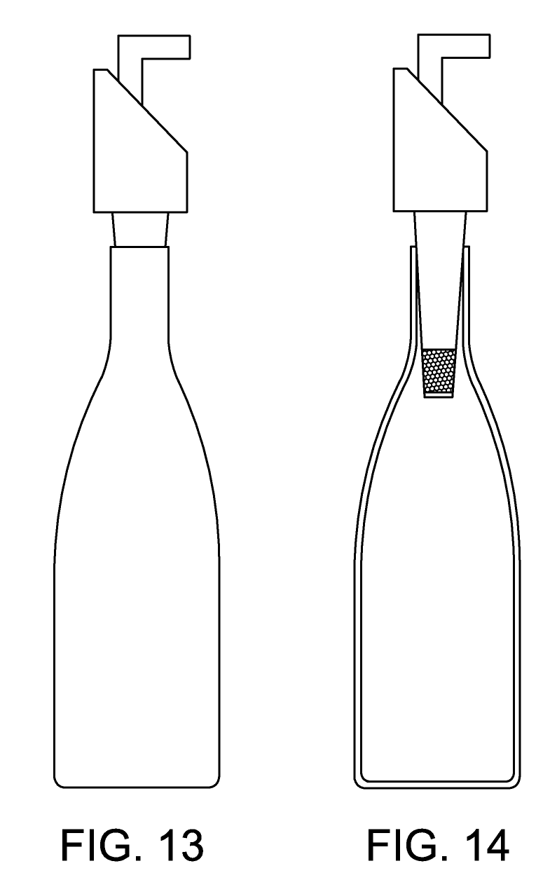

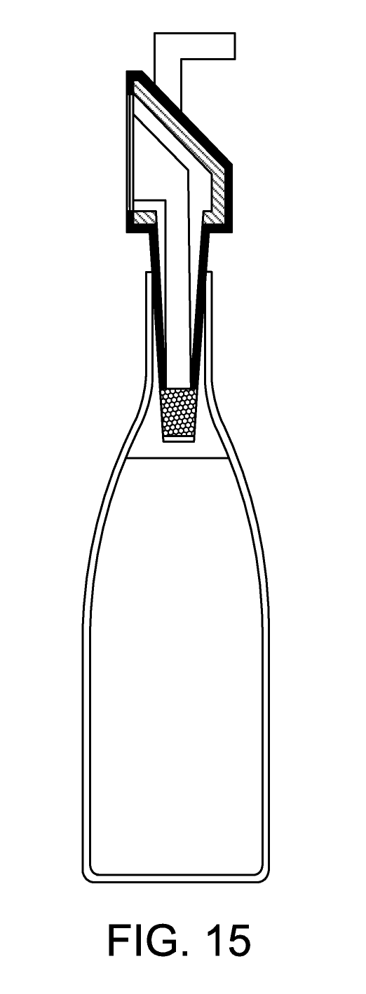

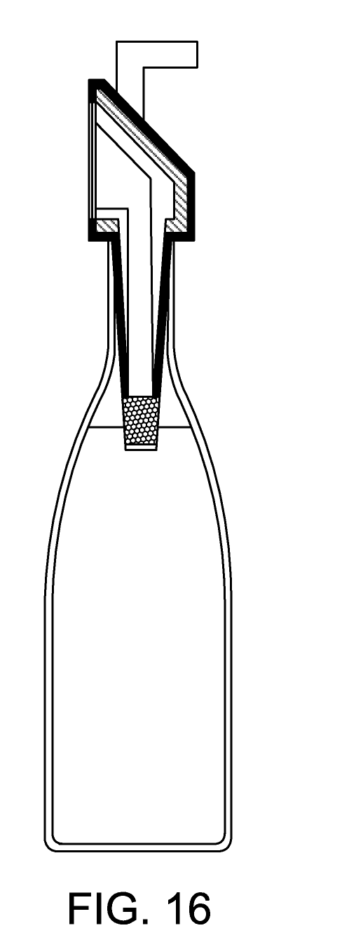

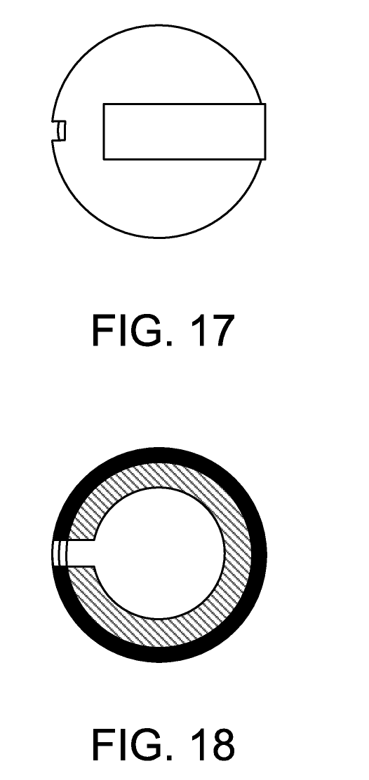

In another embodiment, as shown in FIGS. 13-18, the band may be omitted from the device. In one embodiment, the open position is associated with a first length of the device passing through an opening of the container and the closed position is associated with a second length of the device passing through the opening of the container, wherein the second length is greater than the first length.

In one embodiment, the container may be shaken, tipped, or otherwise moved so that fluid stored within the container, or vapor emanating from the fluid, comes into contact with the freshness sensor.

In an exemplary embodiment, a beverage diagnostic device includes a body forming a fluid chamber, and a freshness sensor, where the body is configured to couple to a beverage container that contains a fluid. In one aspect, the freshness sensor is a timer. In one aspect, the device includes a timer activator that activates the timer when the device is coupled to the beverage container. In one aspect, the timer is a mechanical timer, an electrical timer, or any combination thereof. In one aspect, the body comprises an upper portion and a lower portion, where a fluid passage is formed within the body from the lower portion to a spout located on the upper portion of the device. In one aspect the freshness sensor is a filter located on a lower portion of the device, and the lower portion of the device is configured to contact the fluid.

In an exemplary embodiment, a method of determining freshness of a fluid stored within a container includes coupling a beverage diagnostic device to the container and using a freshness sensor to measure the freshness of the fluid stored within the container. In one aspect, the beverage diagnostic device includes a body forming a fluid chamber and a freshness sensor, where the body is configured to couple to a beverage container that contains a fluid. In one aspect, the freshness sensor is a timer. In one aspect, the beverage diagnostic device further comprises a timer activator that activates the timer when the device is coupled to the beverage container.

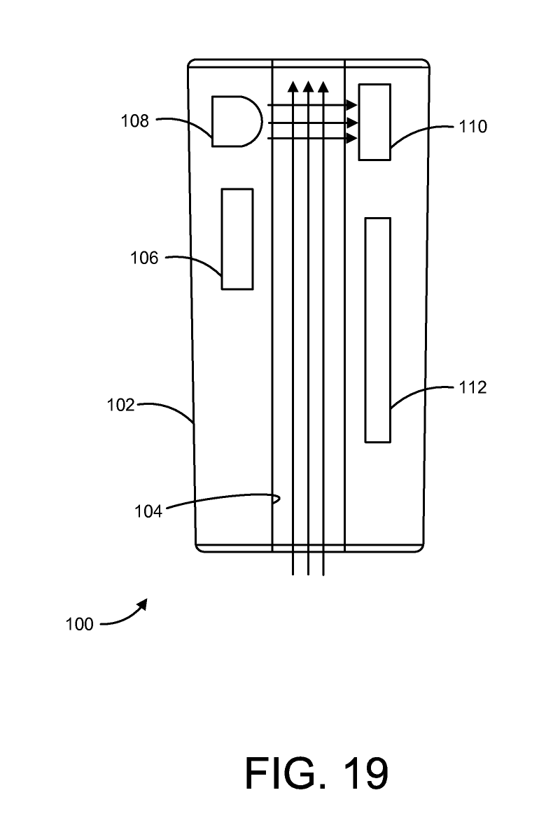

In an exemplary embodiment, as illustrated in FIG. 19 with continuing reference to FIGS. 1-18, a wine spout is generally referred to by the reference numeral 100 and includes a stopper or body member 102, at least a lower portion of which is adapted to be disposed in, or connected to, a wine bottle. In an exemplary embodiment, at least a lower portion of the body member 102 is adapted to be inserted through the mouth of a wine bottle, and extend within the neck of the wine bottle. A longitudinally-extending bore 104 is formed through the body member 102. An electrical power source, such as a battery 106, is connected to the body member 102. An emitter 108, a sensor 110, and a circuit board 112 are also connected to the body member 102. Each of the emitter 108, the sensor 110, and the circuit board 112 is in electrical communication with the battery 106. The circuit board 112 is in electrical communication with each of the emitter 108 and the sensor 110. The emitter 108 and the sensor 110 are opposed to one another across the bore 104. In an exemplary embodiment, the circuit board 112 includes an accelerometer, which functions as a switch as described in further detail below. In several exemplary embodiments, the wine spout 100 includes an indicator or output device, such as a digital display, one or more lights, one or more alarms, or any combination thereof. In several exemplary embodiments, the indicator or output device provides visual, auditory, another type of sensory feedback, or any combination thereof.

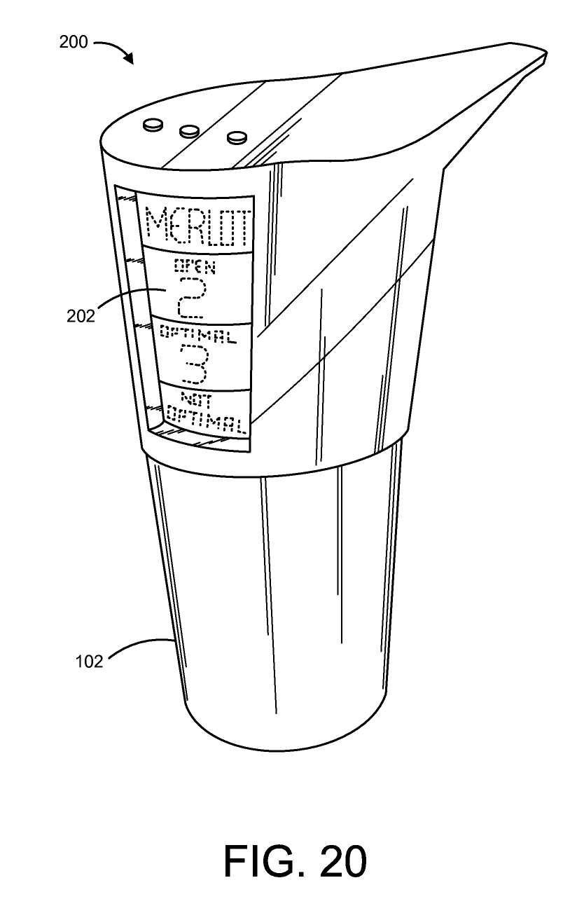

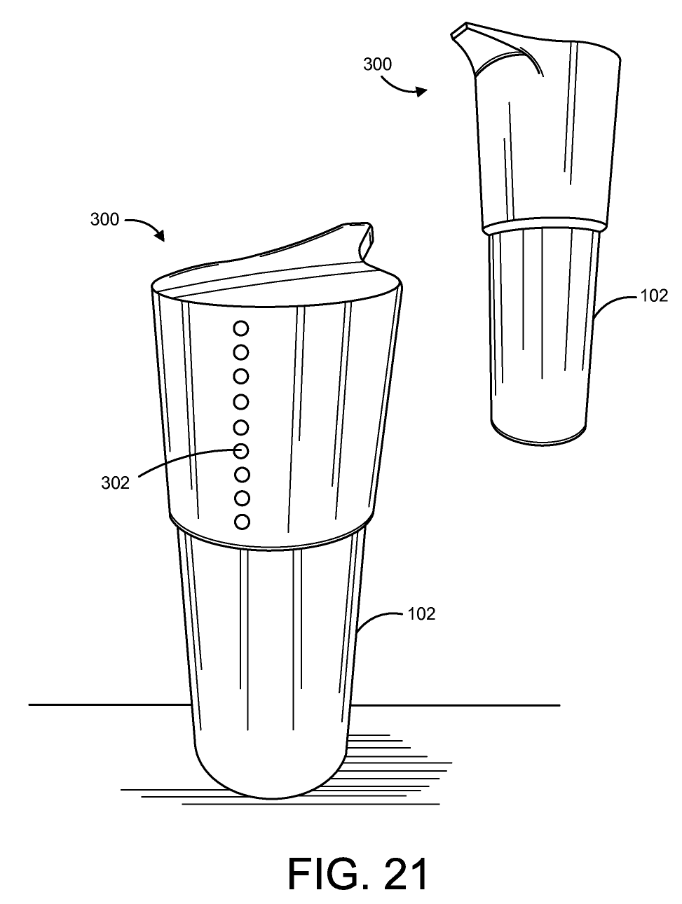

In addition to the above-described components, the wine spout 100 may include other components, such as components shown in FIG. 20 and/or FIG. 21, and/or FIGS. 1-19.

In operation, in an exemplary embodiment, the stopper or body member 102 is inserted, through the mouth of a wine bottle that contains wine, so that at least the lower portion of the body member 102 extends within the neck of the wine bottle. In an exemplary embodiment, the wine spout 100 is coupled or connected to the wine bottle as a result of the extension of the body member 102 within the neck of the wine bottle.

After the wine spout 100 is coupled to the wine bottle, the wine bottle is moved and/or rotated so that at least some of the wine flows through the bore 104.

The accelerometer on the circuit board 112 detects this movement/rotation of the wine bottle. As a result of this detection, the wine spout 100 is turned on, that is, electrical power is supplied to the emitter 108 and the sensor 110. Thus, the accelerometer is part of a switch, the activation of which causes electrical power to be supplied to the wine spout 100. Once activated, the emitter 108 emits a wavelength of light or a variety of wavelengths in a variety of spectral ranges. The reaction or interaction between the wavelengths and the flowing wine is detected by the sensor 110. The sensor 110 sends one or more signals to the circuit board 112, which operates to diagnose the wine, that is, determine if the wine is suitable for consumption or spoiled. The circuit board 112 sends one or more signals to the indicator or output device, which communicates the state of the wine.

In an exemplary embodiment, the emitter 108 and the sensor 110 are Paired Emitting Detecting Diodes (PEDD). The emitter 108 is a light emitting diode that emits a specified wavelength. The sensor 110 is a photodiode positioned opposite the emitter 108 across the bore 104. During operation, the wavelength emitted by the emitter 108 reacts with free sulfur dioxide (SO₂) in the wine and the reaction is detected by the sensor 110. The sensor 110 sends one or more signals to the circuit board 112, which determines the amount of free sulfur dioxide in the wine, which is correlated with good or bad wine. The circuit board 112 sends one or more signals to the indicator or output device, which communicates the state of the wine. In this exemplary embodiment, free sulfur dioxide in the wine is used as an indicator to determine whether the wine is good or bad. In an exemplary embodiment, the sensor 110 (or photodiode) detects the spectral response that correlates to the level of sulfur dioxide present in the wine; the circuit board 112 correlates the spectral response with an oxidation level to determine if the wine is suitable to consume.

In another exemplary embodiment, the emitter 108 emits a specified wavelength of light, which is passed through the wine in the bore 104. The intensity of the wavelength of light is detected by the sensor 110 (which may be characterized as a detector). The sensor 110 sends one or more signals to the circuit board 112, which determines whether the detected intensity correlates with good or bad wine. The circuit board 112 sends one or more signals to the indicator or output device, which communicates the state of the wine. In this exemplary embodiment, light absorbance in the wine is used as an indicator to determine whether the wine is good or bad.

In an exemplary embodiment, during the operation of the wine spout 100, wine does not flow through the bore 104; instead, the wine bottle remains closed and is flipped over so that static wine is disposed in the bore 104.

In several exemplary embodiments, instead of, or in addition to an accelerometer on the circuit board 112, the wine spout 100 includes one or more other switches to selectively supply electrical power to the above-described electrically powered components. The one or more other switches may include manual, fluid detector, pressure sensor, other types of switches, or any combination thereof.

In several exemplary embodiments, instead of, or in addition to, the circuit board 112, the wine spout 100 includes one or more other controllers, computers, and/or processors, each of which may include or be a part of one or more of the following: a conventional programmable general purpose controller, an application specific integrated circuit (ASIC), other conventional controller devices and/or any combination thereof.

In an exemplary embodiment, as illustrated in FIG. 20 with continuing reference to FIGS. 1-19, a wine spout is referred to by the reference numeral 200 and includes all of the components of the wine spout 100 of FIG. 19, which components are given the same reference numerals. The wine spout 200 includes an indicator or output device in the form of a digital display 202, which communicates the state of the wine. The operation of the wine spout 200 is identical to the operation of the wine spout 100 and therefore the operation of the wine spout 200 will not be described in further detail.

In an exemplary embodiment, as illustrated in FIG. 21 with continuing reference to FIGS. 1-20, a wine spout is referred to by the reference numeral 300 and includes all of the components of the wine spout 100 of FIG. 19, which components are given the same reference numerals. The wine spout 300 includes an indicator or output device in the form of an array of lights 302, which communicates the state of the wine. The operation of the wine spout 300 is identical to the operation of the wine spout 100 and therefore the operation of the wine spout 300 will not be described in further detail.

In several exemplary embodiments, a wine bottle or other wine container may include, or be integral with, one or more of the above-described embodiments of wine spouts or component(s) thereof.

In an exemplary embodiment, a method of diagnosing wine includes measuring or detecting free sulfur dioxide (SO₂) levels in the wine or in the headspace of the wine bottle. In an exemplary embodiment, the method includes emitting a wavelength of light into the wine and detecting a spectral response that correlates to the level of free sulfur dioxide present in the wine. In an exemplary embodiment, the method includes emitting a wavelength of light into the headspace of the wine bottle and detecting a spectral response that correlates to the level of free sulfur dioxide present in the wine.

In an exemplary embodiment, a method of diagnosing wine includes measuring light absorbance in the wine. In an exemplary embodiment, such a method includes a spectrophotometry process.

In an exemplary embodiment, a method of diagnosing the state of wine includes detecting that free sulfur dioxide has been consumed in the wine. In an exemplary embodiment, such a method includes detecting that dissolved oxygen levels in the wine have increased. The increase in dissolved oxygen levels indicates spoilage of the wine. In an exemplary embodiment, a wine spout is connected to a wine bottle or other container; the wine spout is configured to detect dissolved oxygen levels in the wine contained by the wine bottle. In an exemplary embodiment, the wine spout is configured to detect dissolved oxygen levels in the wine contained by the wine bottle as at least a portion of the wine flows through the wine spout. In an exemplary embodiment, a method of diagnosing a beverage includes detecting that free sulfur dioxide has been consumed in the beverage. In an exemplary embodiment, such a method includes detecting that dissolved oxygen levels in the beverage have increased. The increase in dissolved oxygen levels indicates spoilage in the beverage.

In an exemplary embodiment, a method of diagnosing a beverage includes detecting the pH level in the beverage. In an exemplary embodiment, a method of diagnosing wine contained in a wine bottle includes detecting the pH level in the wine.

In an exemplary embodiment, a method of diagnosing wine includes measuring the change in the aroma of the wine. In an exemplary embodiment, such a method includes programming a sensor to respond to various levels of acetaldehyde odors, coupling the sensor to a wine bottle or other wine container (e.g. by way of a diagnostic device as described herein), and using the sensor to detect the aroma change in the wine contained in the wine bottle. In an exemplary embodiment, the sensor is, includes, or is part of, an electronic nose, or incorporates in whole or in part electronic nose technology; in an exemplary embodiment, such electronic nose technology includes an odor reactive polymer sensor array and a pattern recognition system, such as an artificial neural network (ANN), enabling the sensor to process new odors based on a pattern of aromas created by earlier experiences. In an exemplary embodiment, the levels of acetaldehyde are correlated with levels of oxidation in the wine. In an exemplary embodiment, the sensor is part of a wine spout, which is coupled to the wine bottle.

In an exemplary embodiment, a method of diagnosing wine includes detecting or measuring an increase in acetaldehyde in the wine. In an exemplary embodiment, such a method includes programming a sensor to detect increases in acetaldehyde levels, coupling the sensor to a wine bottle or other wine container, and using the sensor to detect the increase in acetaldehyde in the wine contained in the wine bottle. In an exemplary embodiment, the sensor is part of a wine spout, which is coupled to the wine bottle.

In an exemplary embodiment, a method of diagnosing wine includes measuring changes in ethanol levels in the wine or in the headspace of the wine bottle. In an exemplary embodiment, such a method includes providing a sensor to detect changes in ethanol levels, coupling the sensor to a wine bottle or other wine container, and using the sensor to detect the changes in ethanol levels in the wine contained in the wine bottle. In an exemplary embodiment, such a method includes providing a sensor to detect changes in ethanol levels, coupling the sensor to a wine bottle or other wine container, and using the sensor to detect the changes in ethanol levels in the headspace of the wine bottle. In an exemplary embodiment, the sensor is part of a wine spout, which is coupled to the wine bottle.

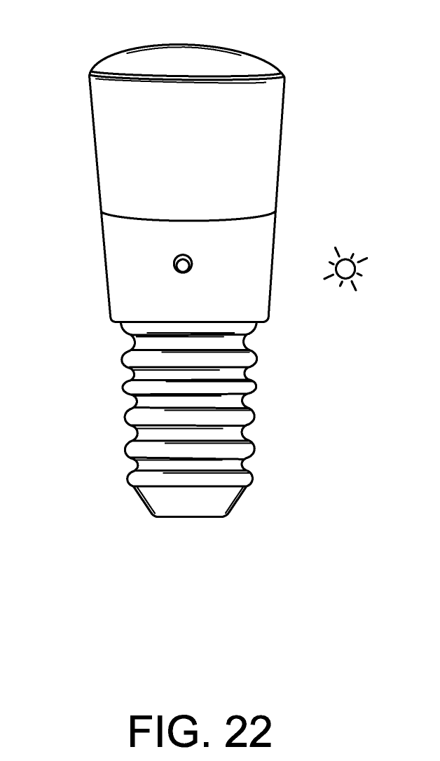

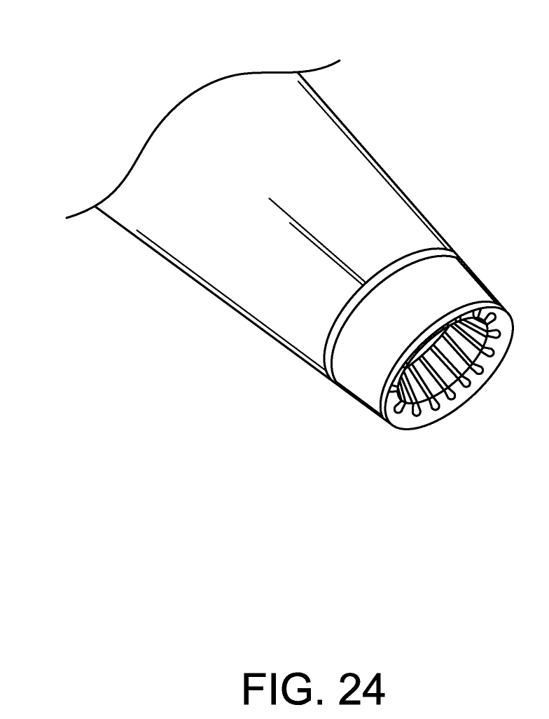

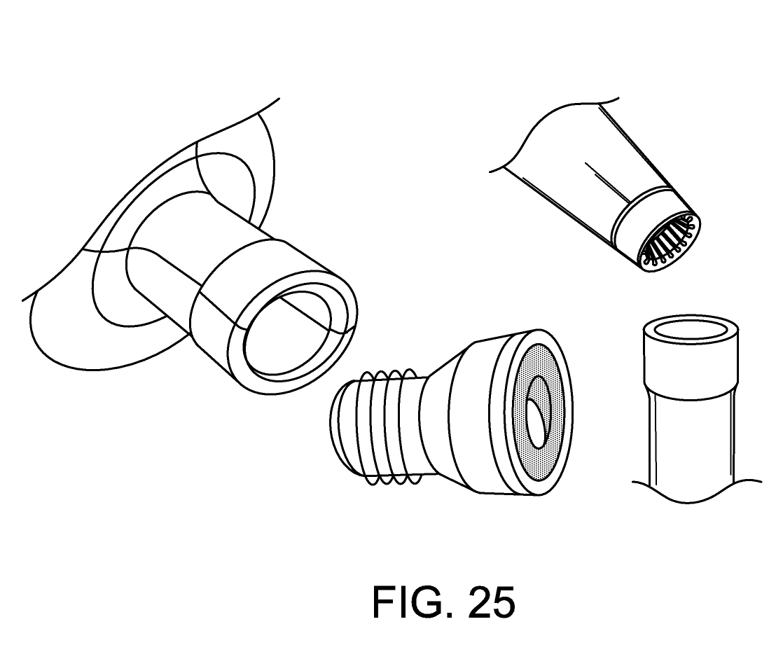

In an exemplary embodiment, as illustrated in FIGS. 22-25 with continuing reference to FIGS. 1-21, a wine bottle stopper includes a housing, a switch connected to one end of the housing, a sealing element connected to the other end of the housing, and a sensor cover connected to the sealing element. A sensor is disposed within the sealing element and/or the sensor cover. A battery and a printed circuit board (PCB) in electrical communication therewith are at least partially disposed within the housing. The switch is in electrical communication with the PCB. The sensor is in electrical communication with the PCB. An LED, other type of light, or other indicator is in electrical communication with the PCB and is positioned proximate the housing and/or the sealing element. The PCB includes one or more controllers, which are in electrical communication with the battery, the sensor, and the LED or other indicator.

In an exemplary embodiment, the wine bottle stopper may be characterized as a replacement cork. In an exemplary embodiment, the switch is a push-button switch, a toggle switch, or any combination thereof. In an exemplary embodiment, the sealing element is a ribbed rubber or other elastomer tubular component adapted to sealingly engage the inside surface of the neck of the wine bottle. The wine bottle stopper can provide a liquid tight and air tight seal within the neck of the wine bottle. In an exemplary embodiment, the sensor includes sniffer technology. In an exemplary embodiment, the sensor includes, or is, a free sulfur dioxide sensor. In several exemplary embodiments, the sensor includes in whole or in part one or more of the following sensors or portions thereof: Sulfur Dioxide Sensor Part Number 008-1113-000, which is available from RAE Systems, Inc., San Jose, Calif.; Sulfur Dioxide Sensor SKU Number OXA-S02, which is available from Variable, Inc., Chattanooga, Tenn.; Sulfur Dioxide Sensor Model DM-700-502, which is available from Detcon, Inc., The Woodlands, Tex.; and S02-D4 Sulfur Dioxide Sensor, which is available from Alphasense Ltd, Great Notley, United Kingdom.

In operation, in an exemplary embodiment, with continuing reference to FIGS. 22-25, a cork or cap is initially removed from a wine bottle (i.e., the wine bottle is opened). When it is desired to close and seal the wine bottle, the wine bottle stopper of FIGS. 22-25 is used. More particularly, the sealing element is inserted into the wine bottle and sealingly engages the inside surface of the wine bottle neck. Thus, the wine bottle stopper replaces the cork. When it is desired to drink additional wine, the switch is activated, causing the battery to supply electrical power to the sensor via the PCB. The sensor detects whether there is sulfur dioxide in the head space, that is, the space within the wine bottle that extends between the liquid and the wine bottle stopper. If the sensor does detect sulfur dioxide in the headspace (e.g. the sensor detects a level of sulfur dioxide above a predetermined threshold level), the sensor transmits one or more signals to the PCB, which, in turn, causes the LED or other light to emit light, indicating that the wine is palatable, fresh, or “good.” If the sensor does not detect sulfur dioxide in the headspace, or detects a level of sulfur dioxide that is below a predetermined threshold level, the LED or other light does emit light, indicating that the wine is not palatable, not fresh, or “bad.”

In an exemplary embodiment, electrical power can be continuously provided to the sensor. In an exemplary embodiment, electrical power can be continuously provided to the sensor, and the switch may be omitted.

In an exemplary embodiment, the sensor operates in one or more of the manners described above in connection with FIGS. 1-21.

In an exemplary embodiment, instead of detecting sulfur dioxide in the headspace, the sensor detects acetaldehyde odors.

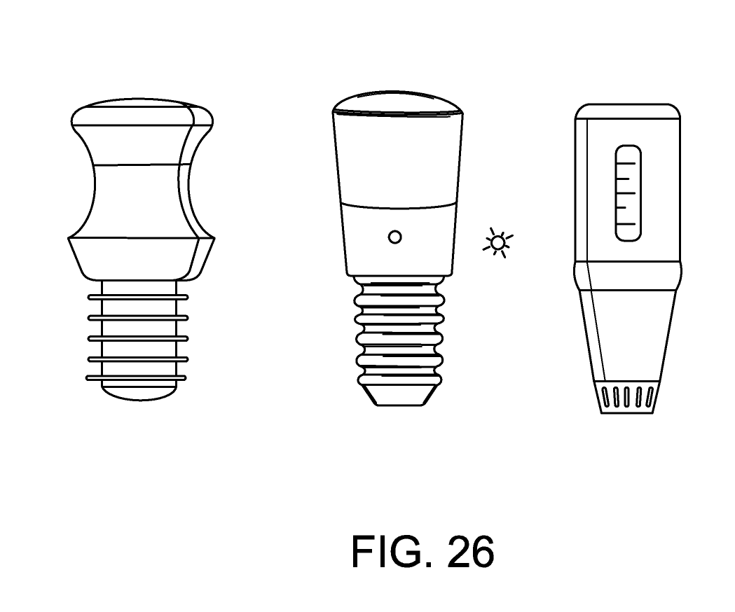

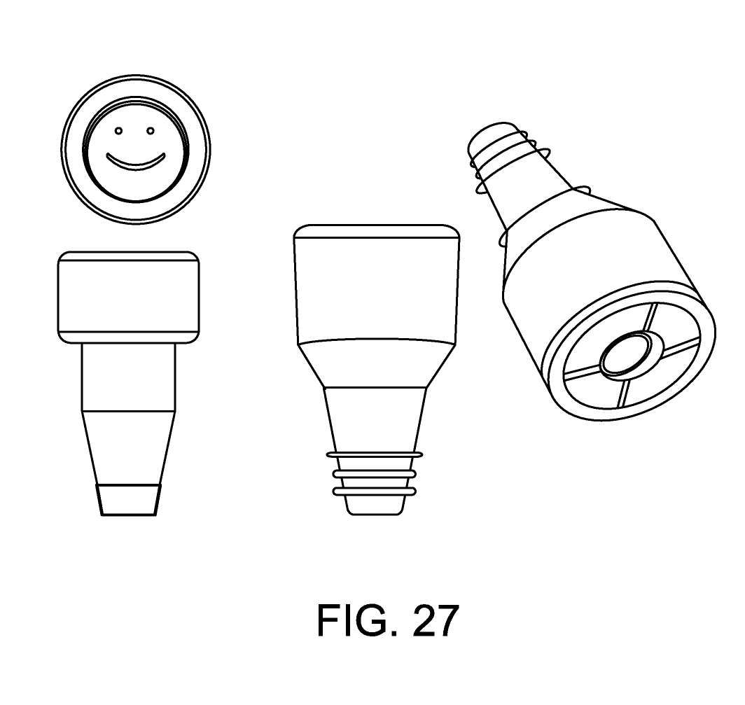

In an exemplary embodiment, as illustrated in FIGS. 26 and 27 with continuing reference to FIGS. 1-25, wine bottle stoppers according to respective exemplary embodiments are provided, each of the wine bottle stoppers being either identical to the wine bottle stopper of FIGS. 22-25, or similar to the wine bottle stopper of FIGS. 22-25 except for differences in the respective shapes (or configurations) of the housing, switch, sealing element, sensor cover, or any combination thereof. Additionally, one of the wine bottle stoppers illustrated in FIG. 26 does not include a single LED or other light; instead, a scale is in electrical communication with the PCB and the scale indicates the degree to which the wine is fresh (bad, good, fresh, etc.). The same wine bottle stopper illustrated in FIG. 26 also does not include a sensor cover.

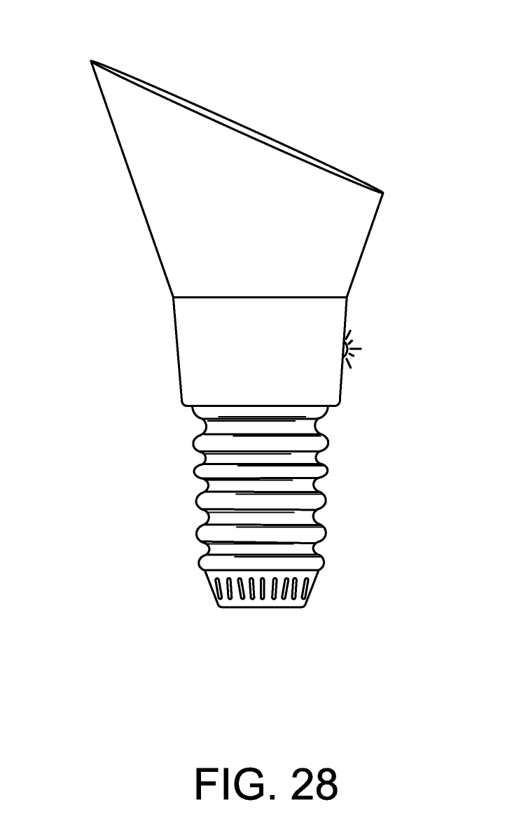

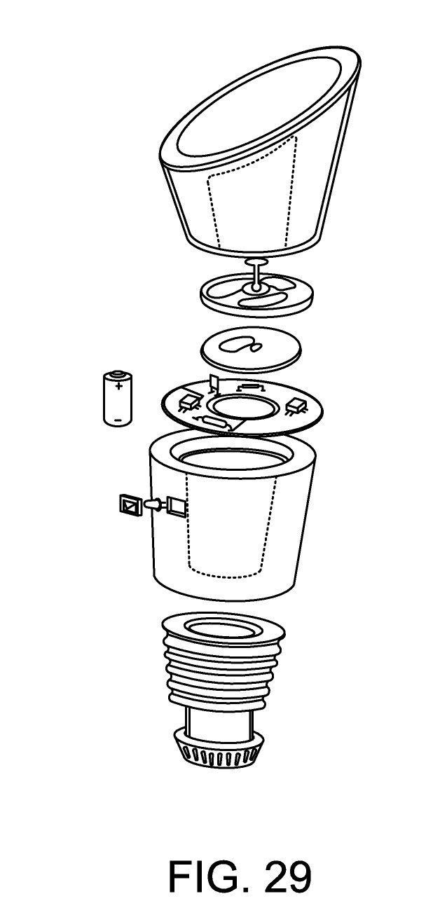

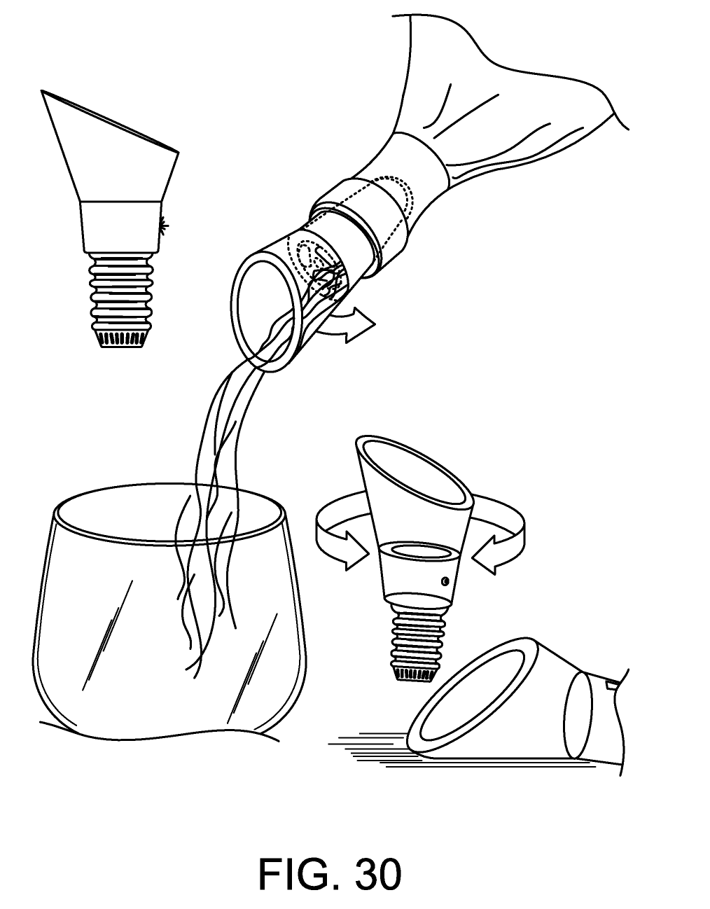

In an exemplary embodiment, as illustrated in FIGS. 28 and 29 with continuing reference to FIGS. 1-27, a wine bottle stopper includes a pour spout (or wine spout), a housing connected to the spout, a sealing element connected to the housing and opposing the spout, a sensor connected to the sealing element and opposing the housing, and an LED, other type of light, or other indicator positioned within or proximate an opening formed in the housing.

As shown in FIG. 29, the wine bottle stopper further includes an opener, a blocker, an annular-shaped PCB, a battery, and a lens. The opener includes a disc component with an opening formed therethrough. The blocker includes a disc component and a plurality of openings formed therethrough. One of the opener and the blocker is connected to the lower end portion of the pour spout, and the other of the opener and the blocker is connected to the upper end portion of the housing. Relative rotation between the pour spout and the housing is permitted; thus, relative rotation between the opener and the blocker is permitted. Each of the battery, the LED or other light, and the sensor is in electrical communication with the PCB. The lens is connected to the housing and covers the LED or other light. At least the PCB, the lens, and the battery are disposed within the housing.

In operation, in an exemplary embodiment, as illustrated in FIG. 30 with continuing reference to FIGS. 1-29, a cork or cap is initially removed from a wine bottle (i.e., the wine bottle is opened). When it is desired to close the wine bottle, the wine bottle stopper of FIGS. 28 and 29 is used. More particularly, the sealing element is inserted into the wine bottle and sealingly engages the inside surface of the wine bottle neck. Thus, the wine bottle stopper replaces the cork. The battery supplies electrical power to the sensor via the PCB. The sensor detects whether there is sulfur dioxide in the headspace, that is, the space within the wine bottle that extends between the liquid and the wine bottle stopper. If the sensor does detect SO₂ in the headspace, the sensor transmits one or more signals to the PCB, which, in turn, causes the LED or other light to emit light, indicating that the wine is palatable, fresh, or “good.” If the sensor does not detect sulfur dioxide in the headspace, the LED or other light does emit light, indicating that the wine is not palatable, not fresh, or “bad.” When it is desired to drink additional wine, the LED or other light is observed to see if it emits light to determine whether the wine is still fresh. If drinking the wine is still desired, as shown in FIG. 30, relative rotation is effected between the pour spout and the housing so that the opening in the opener is at least partially aligned with one of the openings in the blocker. After the respective openings are so aligned, wine is poured out of the wine bottle, flowing through the sensor, the sealing element, the housing, the aligned openings, and the pour spout. In an exemplary embodiment, the pour spout is twisted, relative to the remainder of the wine stopper device and the wine bottle, in order to align the openings.

In several exemplary embodiments, to close the wine bottle, relative rotation between the pour spout and the housing of the wine bottle stopper is effected so that no openings are aligned.

In an exemplary embodiment, the pour spout functions as an aeration device. In an exemplary embodiment, the pour spout includes features configured to promote aeration.

In an exemplary embodiment, instead of detecting SO₂ in the headspace, the sensor detects acetaldehyde odors.

In an exemplary embodiment, the wine bottle stopper of FIGS. 28-30 includes a switch, such as a push button switch or toggle switch, which can be activated to cause the battery to supply electrical power to the sensor via the PCB.

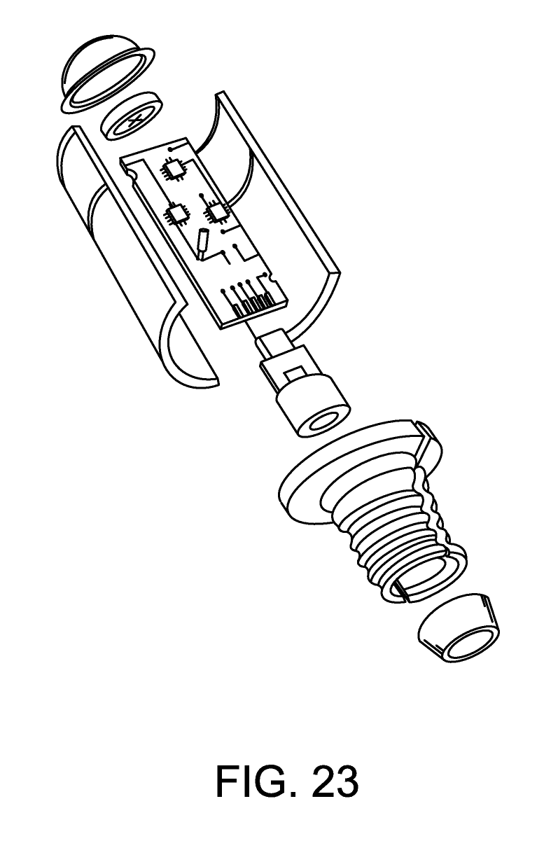

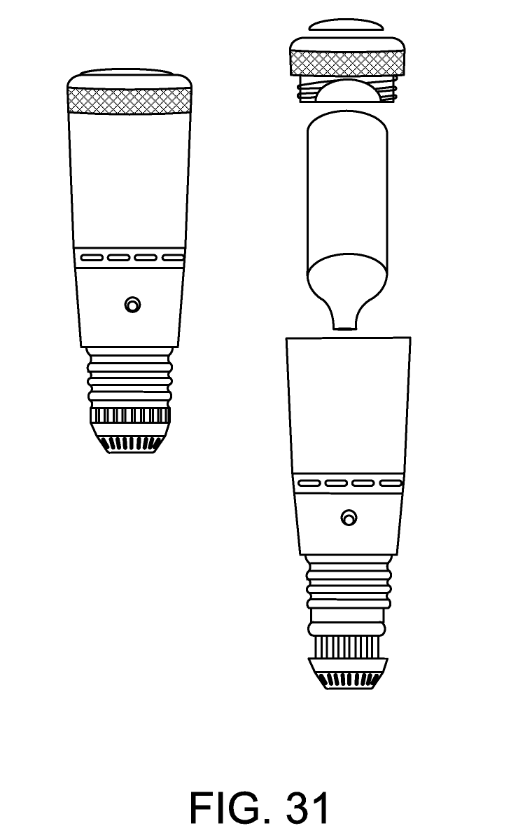

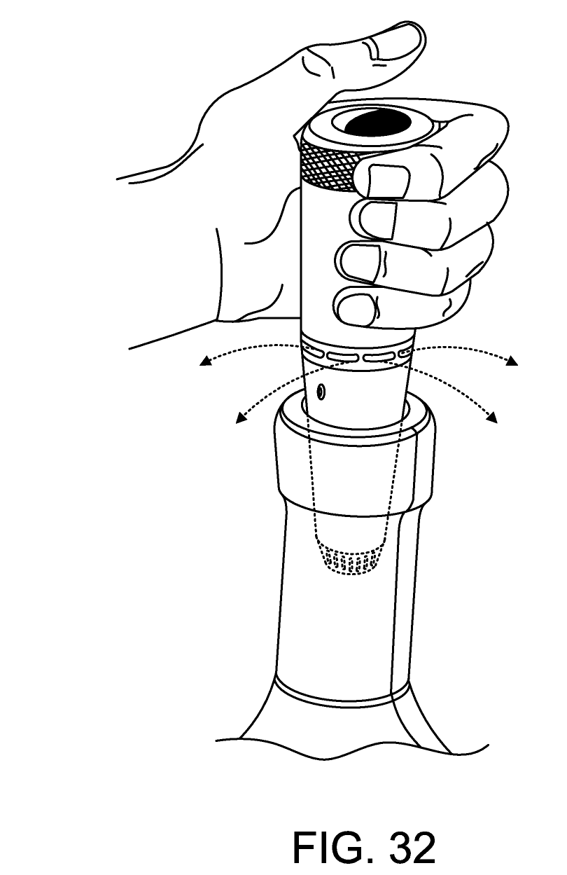

In an exemplary embodiment, as illustrated in FIGS. 31 and 32 with continuing reference to FIGS. 1-30, a wine bottle stopper includes components that are similar to the wine bottle stopper of FIGS. 22 and 23. The sensor cover may be omitted from the wine bottle stopper of FIGS. 31 and 32. Additionally, as shown in FIGS. 31 and 32, a plurality of circumferentially-spaced openings are formed at the upper end portion of the housing. A tubular member is connected to the upper end portion of the housing. A pressure vessel, or pressurized argon cartridge, is disposed within the internal region of the tubular member. The internal region of the tubular member is adapted to be in fluid communication with the internal region of the wine bottle. A top cap is connected to the upper end portion of the tubular member. A button is connected to the top cap and is operably coupled to the argon cartridge.

In operation, in an exemplary embodiment, with continuing reference to FIGS. 31 and 32 (as well as to FIGS. 1-30), with respect to freshness detection, the wine bottle stopper of FIGS. 31 and 32 operates in a manner identical to the manner by which the wine bottle stopper of FIGS. 22 and 23 operates to detect whether the wine is palatable, fresh, or “good.”

Additionally, during operation, as shown in FIG. 32, to preserve the freshness of the wine, argon is introduced into the wine bottle when the wine bottle stopper is coupled to the wine bottle. More particularly, the button is depressed, causing the argon cartridge to release argon into the headspace of the wine bottle and forcing oxygen to flow out of the circumferentially-spaced openings. As a result, oxygen is purged from the headspace of the wine bottle, thereby preserving the freshness of the wine. In an exemplary embodiment, each of the circumferentially-spaced openings includes a one-way valve operably coupled thereto; each of the one-way valves permits the oxygen to flow out of the headspace via the corresponding opening, but does not permit any gases to flow into the headspace via the opening.

In several exemplary embodiments, instead of an argon cartridge, the wine bottle stopper of FIGS. 31 and 32 includes a cartridge charged with another type of gas, such as another type of inert gas (e.g., nitrogen).

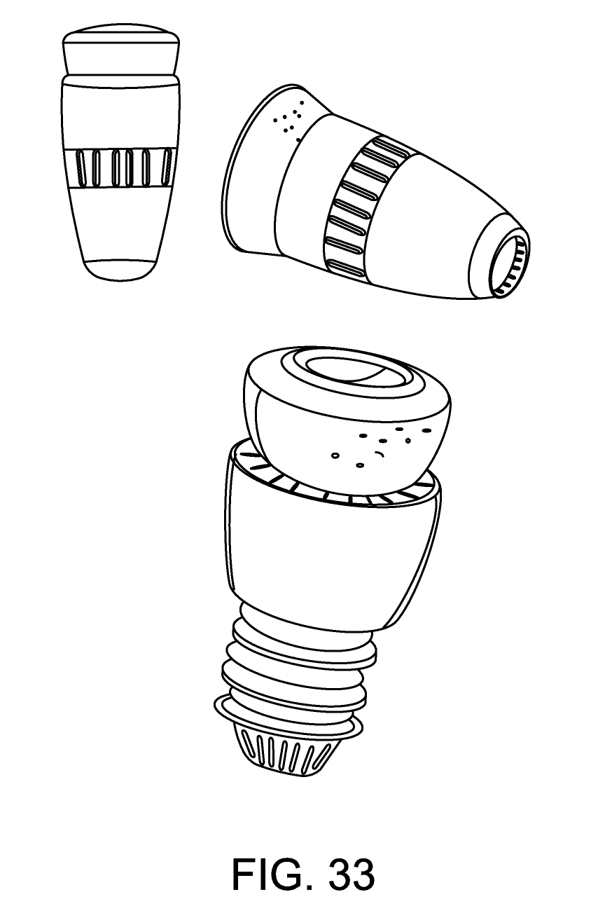

In an exemplary embodiment, as illustrated in FIG. 33 with continuing reference to FIGS. 1-32, one of the illustrated wine bottle stoppers includes a sensor, a housing connected to the sensor and including sealing features, a plurality of circumferentially-spaced openings formed in the housing, and a pressurized cartridge extending into the housing, the cartridge including a button. The other of the illustrated wine bottle stoppers includes a sensor, a sealing element connected to the sensor, a housing connected to the sealing element and defining an axially-facing surface, a plurality of circumferentially-spaced openings formed in the axially-facing surface, and a pressurized cartridge extending into the housing, the cartridge including a button. In several exemplary embodiments, the operation of each of the wine bottle stoppers illustrated in FIG. 33 is substantially similar to the operation of the wine bottle stopper of FIGS. 31 and 32 and therefore the operation of each of the wine bottle stoppers illustrated in FIG. 33 will not be described in detail.

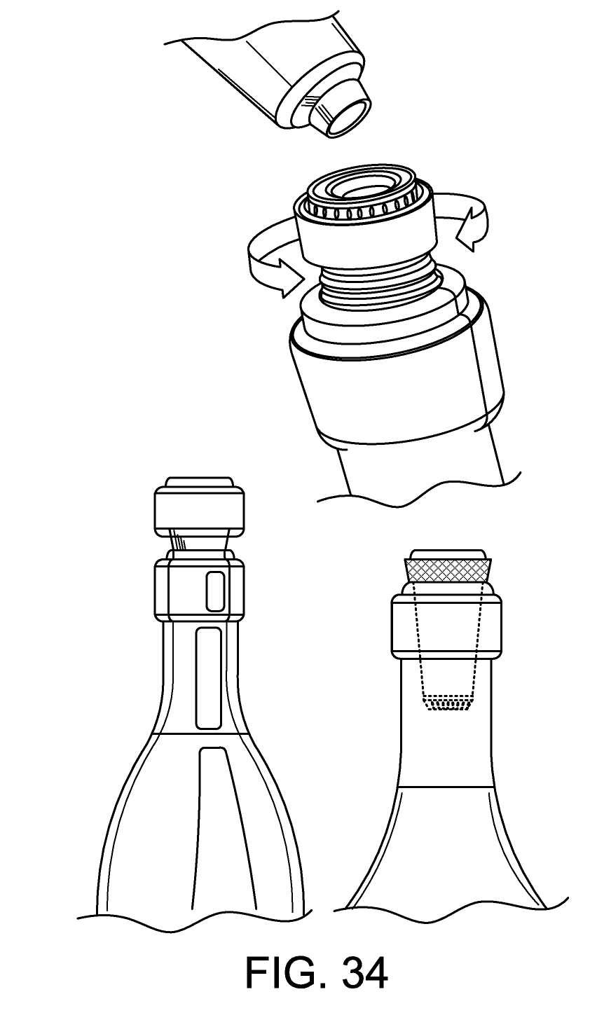

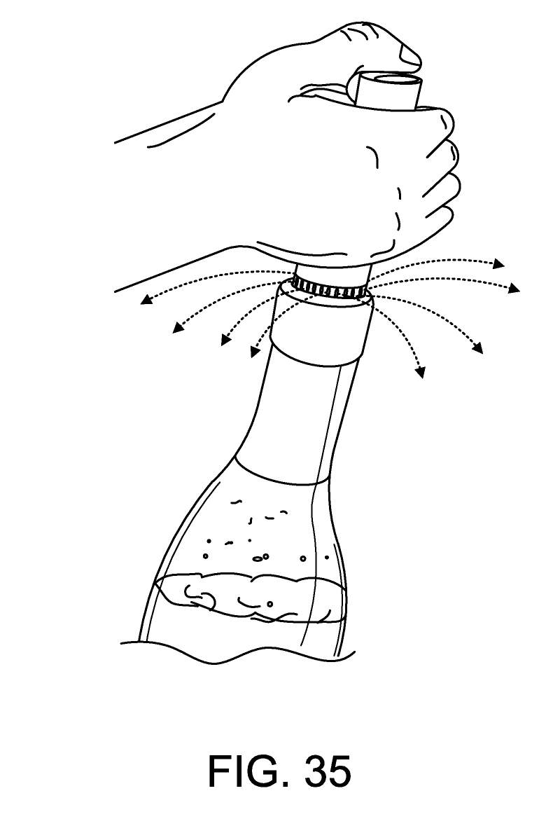

In an exemplary embodiment, as illustrated in FIGS. 34 and 35 with continuing reference to FIGS. 1-33, a system includes a wine bottle stopper and a preservation device. The wine bottle stopper of FIGS. 34 and 35 includes components that are similar to the components of the wine bottle stopper of FIGS. 22-25. Additionally, the housing of the wine bottle stopper of FIGS. 34 and 35 includes a one-way valve at the top thereof. A plurality of circumferentially-spaced openings are formed at the upper end portion of the housing. The housing is rotatable, relative to at least the sealing element and the sensor.

The preservation device is a portable, handheld device. The preservation device includes a housing and an adapter at one end thereof. An argon cartridge is disposed in the housing and is adapted to release pressurized argon via the adapter. In several exemplary embodiments, instead of argon, the housing includes a cartridge charged with another type of gas, such as another type of inert gas (e.g., nitrogen). In several exemplary embodiments, the cartridge is omitted and the charged gas is disposed in the housing, contacting the inside surface of the housing. In an exemplary embodiment, the preservation device includes a switch connected to the housing.

In operation, in an exemplary embodiment, with continuing reference to FIGS. 34 and 35 (as well as to FIGS. 1-33), with respect to freshness detection, the wine bottle stopper of FIGS. 34 and 35 operates in a manner identical to the manner by which the wine bottle stopper of FIGS. 22 and 23 operates to detect whether the wine is palatable, fresh, or “good.” To pour the wine out of the bottle, the housing is twisted relative to at least the sealing element, causing a through-passage to form in the wine bottle stopper and allowing the wine to be poured out through the wine bottle stopper. After the wine has been poured, to close the wine bottle, the housing is again twisted relative to at least the sealing element, removing the through-passage.

Additionally, during operation, as shown in FIG. 35, to preserve the freshness of the wine, the preservation device is temporarily coupled to the wine bottle stopper when the wine bottle stopper is installed on the wine bottle. The adapter of the preservation device extends into and/or engages the one-way valve of the housing of the wine bottle stopper. The switch on the housing of the preservation device is activated, causing the argon cartridge to release pressurized argon, which flows through the adapter and the one-way valve and into the headspace of the wine bottle. As a result, oxygen in the headspace is forced to flow out of the headspace via the circumferentially-spaced openings formed in the housing of the wine bottle stopper. As a result, oxygen is purged from the headspace of the wine bottle, thereby preserving the freshness of the wine. The preservation device is then decoupled from the wine bottle stopper by pulling the adapter out of, and/or disengaging the adapter from, the one-way valve of the wine bottle stopper. The one-way valve at the top of the housing of the wine bottle stopper permits the argon to flow into the headspace, but does not permit backflow of any fluid, including any oxygen.

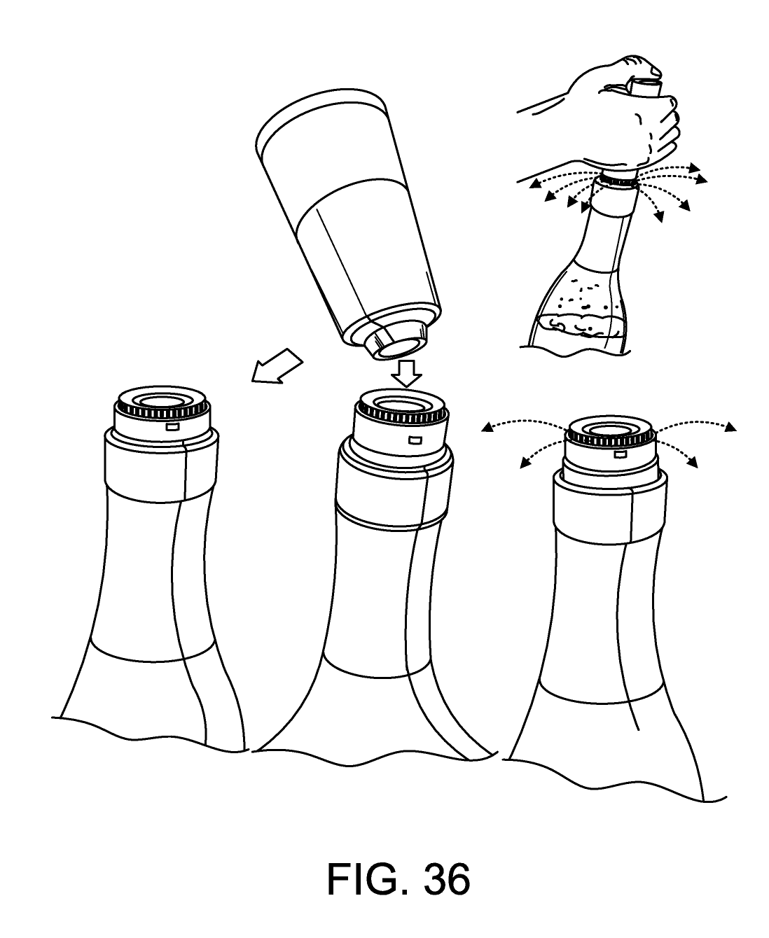

In an exemplary embodiment, as illustrated in FIG. 36 with continuing reference to FIGS. 1-35, a system includes the system of FIGS. 34 and 35, as well as additional wine bottle stoppers, each of which is substantially identical to the wine bottle stopper of the system of FIGS. 34 and 35.

With respect to freshness detection, each of the wine bottle stoppers of the system of FIG. 36 operates in a manner identical to the manner in which the wine bottle stopper of the system of FIGS. 34 and 35 operates. With respect to preservation, the preservation device of FIG. 36 (which is the preservation device of FIGS. 34 and 35) may be temporarily coupled to each of the wine bottle stoppers in the system of FIG. 36, in order to preserve the freshness of the wine contained in the respective wine bottles to which the wine bottle stoppers are coupled. The preservation operation for each wine bottle is identical to the above-described preservation operation of the system of FIGS. 34 and 35. The system of FIG. 36 enables one to purge several bottles of wine effortlessly.

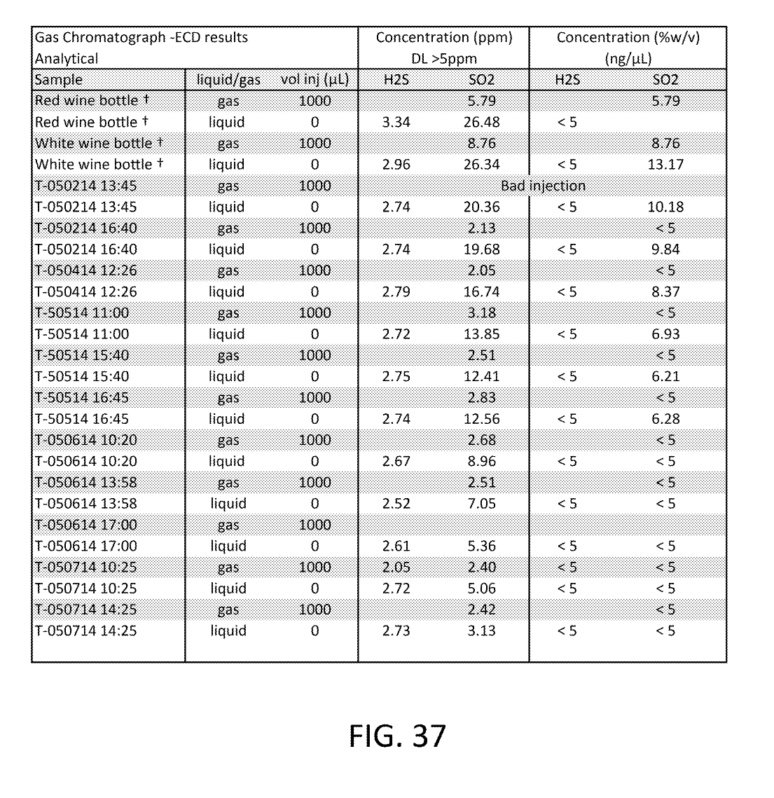

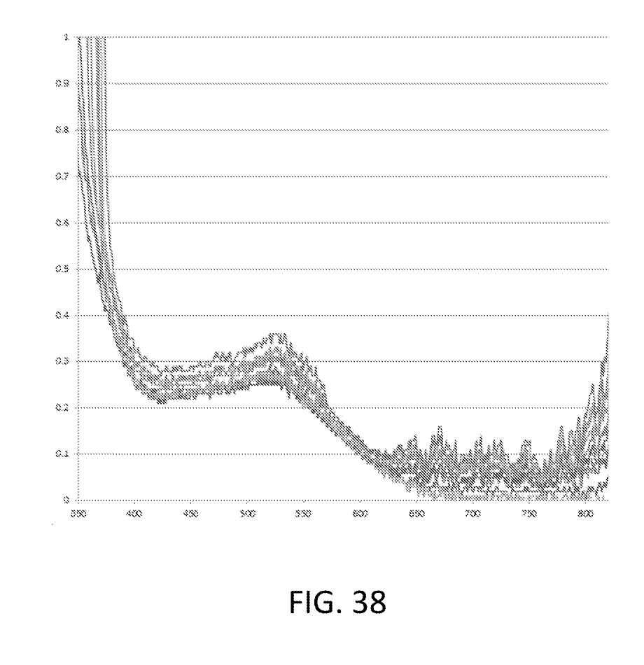

FIG. 37 shows test results for an experiment involving measuring sulfur dioxide (SO₂) and hydrogen sulfide (H₂S) in two bottles of wine. The data was collected using a gas chromatograph detector with an electron capture detector (ECD). The first four line items in the spreadsheet show concentrations of sulfur dioxide and hydrogen sulfide in an unopened bottle of red wine and an unopened bottle of white wine. To allow data to be collected without opening the bottles of wine, samples were collected by inserting a syringe through each bottle's cork. As is shown in the table, prior to opening the bottles, the liquid concentration of SO₂ was about 26 ppm for both bottles of wine, whereas the gas concentration of SO₂ in the headspace was only about 6-9 ppm. Once the bottle of red wine was opened, the concentration of SO₂ in the headspace dropped off sharply below the detection limit of the instrument while the concentration of SO₂ in the liquid gradually declined. In one example, a method for determining wine quality can include measuring a concentration of SO₂ in liquid wine and determining the freshness of the wine based on the measured concentration of SO₂. The wine may be deemed fresh (i.e. good) if the concentration of SO₂ in the liquid is greater than about 5, 10, 15, 20, or 25 ppm. The wine may be deemed not fresh (i.e. bad) if the concentration of SO₂ in the liquid is less than about 5, 10, 15, 20, or 25 ppm.

Experimental testing revealed that absorbance of the wines decreased with time after they were opened. A method for determining wine freshness can include measuring absorbance of wine in a container, and determining freshness of the wine based on the absorbance. In one example, a method can include measuring the absorbance of wine at about 400-600, 450-550, 500-550, or 520 nm, and determining freshness of the wine based on the absorbance. In one example a device for determining wine freshness can include a sensor capable of measuring absorbance at about 400-600, 450-550, 500-550, or 520 nm.

In some examples, in addition to detecting if the wine is good or bad, the device described herein may also detect if the wine is suitable for use as an ingredient in some other entrée or beverage, such as Sangria. In one example, the device may indicate that the wine is suitable for use as an ingredient about 4, 8, 12, or 24 hours before the wine is deemed bad.

In several exemplary embodiments, one or more of the above-described sensors employ in whole or in part sniffer technology, and/or are adapted to detect acetaldehyde odors.

In several exemplary embodiments, one or more of the exemplary embodiments of the present application are provided in whole or in part as described and illustrated in APPENDIX A.

In several exemplary embodiments, one or more of the exemplary embodiments described and illustrated in APPENDIX A are combined in whole or in part with one or more of the other exemplary embodiments described and illustrated in APPENDIX A.

The present disclosure introduces a device according to one or more aspects of the present disclosure.

The present disclosure also introduces a method according to one or more aspects of the present disclosure.

The present disclosure also introduces an apparatus according to one or more aspects of the present disclosure.

The present disclosure also introduces a system according to one or more aspects of the present disclosure.

The present disclosure also introduces a kit according to one or more aspects of the present disclosure.

The present disclosure also introduces a wine spout according to one or more aspects of the present disclosure.

The present disclosure also introduces a wine container according to one or more aspects of the present disclosure.

The present disclosure also introduces a wine bottle stopper according to one or more aspects of the present disclosure.

The present disclosure also introduces a preservation device according to one or more aspects of the present disclosure.

It is understood that variations may be made in the foregoing without departing from the scope of the disclosure. For example, instead of, or in addition to, wine, the foregoing may be applied to other beverages. In several exemplary embodiments, the elements and teachings of the various illustrative exemplary embodiments may be combined in whole or in part in some or all of the illustrative exemplary embodiments. In addition, one or more of the elements and teachings of the various illustrative exemplary embodiments may be omitted, at least in part, and/or combined, at least in part, with one or more of the other elements and teachings of the various illustrative embodiments.

Although a glass bottle is shown in the figures as an example of a beverage container, it is not limiting. Other suitable containers can include plastic bladders, plastic bottles, and aluminum bottles. The devices and method described herein can be modified to accommodate a wide variety of containers.

Any spatial references such as, for example, “upper,” “lower,” “above,” “below,” “between,” “bottom,” “vertical,” “horizontal,” “angular,” “upwards,” “downwards,” “side-to-side,” “left-to-right,” “left,” “right,” “right-to-left,” “top-to-bottom,” “bottom-to-top,” “top,” “bottom,” “bottom-up,” “top-down,” etc., are for the purpose of illustration only and do not limit the specific orientation or location of the structure described above.

In several exemplary embodiments, while different steps, processes, and procedures are described as appearing as distinct acts, one or more of the steps, one or more of the processes, and/or one or more of the procedures may also be performed in different orders, simultaneously and/or sequentially. In several exemplary embodiments, the steps, processes and/or procedures may be merged into one or more steps, processes and/or procedures. In several exemplary embodiments, one or more of the operational steps in each embodiment may be omitted. Moreover, in some instances, some features of the present disclosure may be employed without a corresponding use of the other features. Moreover, one or more of the above-described embodiments and/or variations may be combined in whole or in part with any one or more of the other above-described embodiments and/or variations.

Although several exemplary embodiments have been described in detail above, and/or described in APPENDIX A, the embodiments described are exemplary only and are not limiting, and those skilled in the art will readily appreciate that many other modifications, changes and/or substitutions are possible in the exemplary embodiments without materially departing from the novel teachings and advantages of the present disclosure. Accordingly, all such modifications, changes and/or substitutions are intended to be included within the scope of this disclosure as defined in the following claims. In the claims, means-plus-function clauses are intended to cover the structures described herein as performing the recited function and not only structural equivalents, but also equivalent structures.

What is claimed is:

1. A method for determining freshness of wine, the method comprising:
measuring a lower explosive limit of gas located within a headspace of a container; and
determining freshness of wine in the container based on the lower explosive limit.

2. The method of claim 1, wherein the freshness of the wine is deemed fresh if the lower explosive limit of gas located in the headspace of the container is below about 5%, 10%, or 15%.

3. The method of claim 1, wherein the freshness of the wine is deemed not fresh if the lower explosive limit of gas located in the headspace of the container is above about 5%, 10%, or 15%.

4. The method of claim 1, wherein the lower explosive limit is measured with a photoionization detector, semiconductor, or metal oxide sensor.

5. A method for determining freshness of wine, the method comprising:
measuring a concentration of SO₂ in liquid wine in a container; and
determining the freshness of the wine based on the concentration of SO₂.

6. The method of claim 5, wherein the freshness of the wine is deemed fresh if the concentration of SO₂ in the liquid is greater than about 5, 10, 15, 20, or 25 ppm.

7. The method of claim 5, wherein the freshness of the wine is deemed not fresh if the concentration of SO₂ in the liquid is less than about 5, 10, 15, 20, or 25 ppm.

8. The method of claim 5, further comprising:
measuring a lower explosive limit of gas located within a headspace of the container; and
determining freshness of wine in the container based on the lower explosive limit.

9. The method of claim 8, wherein the freshness of the wine is deemed fresh if the concentration of SO₂ in the liquid is greater than about 5, 10, 15, 20, or 25 ppm and if the lower explosive limit of gas located in the headspace of the container is below about 5%, 10%, or 15%.

10. The method of claim 8, wherein the freshness of the wine is deemed not fresh if the concentration of SO₂ in the liquid is less than about 5, 10, 15, 20, or 25 ppm and if the lower explosive limit of gas located in the headspace of the container is above about 5%, 10%, or 15%.

11. A device according to one or more aspects of the present disclosure.

12. The device of claim 11, further comprising a sensor for detecting a concentration of SO₂ in a liquid, wherein the sensor is capable of detecting a concentration of SO₂ of about 1-35, 1-30, 1-25, 2-30, or 3-30 ppm.

13. The device of claim 11, further comprising a sensor for detecting a concentration of SO₂ in a gas, wherein the sensor is capable of detecting a concentration of SO₂ of about 1-15, 1-10, 2-10, 3-10 ppm.

14. The device of claim 11, further comprising:
a first sensor for detecting a concentration of SO₂ in a liquid, wherein the first sensor is capable of detecting a concentration of SO₂ of about 1-35, 1-30, 1-25, 2-30, or 3-30 ppm; and
a second sensor for detecting a concentration of SO₂ in a gas, wherein the second sensor is capable of detecting a concentration of SO₂ of about 1-15, 1-10, 2-10, 3-10 ppm.

15. The device of claim 11, further comprising a sensor for detecting a lower explosive limit of a gas, wherein the sensor is capable of detecting a lower explosive limit of about 0-30%, 0-25%, or 5-15%.

16. The device of claim 15, wherein the sensor is a photoionization detector, semiconductor, or metal oxide sensor.

17. The device of claim 11, further comprising a sensor for detecting ethanol.

18. The device of claim 11, further comprising:
a first sensor for detecting a lower explosive limit of a gas, wherein the sensor is capable of detecting a lower explosive limit of about 0-30%, 0-25%, or 5-15%; and
a second sensor for detecting a concentration of SO₂ in a liquid, wherein the second sensor is capable of detecting a concentration of SO₂ of about 1-35, 1-30, 1-25, 2-30, or 3-30 ppm.

19. The device of claim 11, further comprising a sensor for detecting absorbance, wherein the sensor is capable of measuring absorbance at about 400-600, 450-550, 500-550, or 520 nm.

20. The device of claim 11, further comprising:
a first sensor for detecting ethanol; and
a second sensor for detecting a concentration of SO₂ in a liquid, wherein the second sensor is capable of detecting a concentration of SO₂ of about 1-35, 1-30, 1-25, 2-30, or 3-30 ppm.
